SEQUENCE LISTING (11 sequences)

SEQ ID NO: 1 - DNA, 21 bp, Homo sapiens
tgagcctgcggggccatggag

SEQ ID NO: 2 - DNA, 21 bp, Homo sapiens
gaggaggtgcacagcaggtag

SEQ ID NO: 3 - DNA, 21 bp, Homo sapiens
ctacctgctgtgcacctcctc

SEQ ID NO: 4 - DNA, 22 bp, Homo sapiens
gcagtgctctagagagctgtgg

SEQ ID NO: 5 - DNA, 42 bp, Rattus sp.
ctctctagagcactgctggaagatgggctgcaccgtgagcgc

SEQ ID NO: 6 - DNA, 24 bp, Rattus sp.
tcagaagaggccacagtccttcag

SEQ ID NO: 7 - DNA, 2403 bp, Artificial
  human/rat chimeric sequence
atggagcgcgcgccgcccgacgggccgctgaacgcttcgggggcgctggcgggcgatgcggcggcggcgggcggggcgcgcggcttctcggcagcctggaccgcggtgctggccgcgctcatggcgctgctcatcgtggccacggtgctgggcaacgcgctggtcatgctcgccttcgtggccgactcgagcctccgcacccagaacaacttcttcctgctcaacctcgccatctccgacttcctcgtcggcgccttctgcatcccactgtatgtaccctacgtgctgacaggccgctggaccttcggccggggcctctgcaagctgtggctggtagtggactacctgctgtgcacctcctctgccttcaacatcgtgctcatcagctacgaccgcttcctgtcggtcacccgagcggtctcataccgggcccagcagggtgacacgcggcgggcagtgcggaagatgctgctggtgtgggtgctggccttcctgctgtacggaccagccatcctgagctgggagtacctgtccgggggcagctccatccccgagggccactgctatgccgagttcttctacaactggtacttcctcatcacggcttccaccctggagttctttacgcccttcctcagcgtcaccttctttaacctcagcatctacctgaacatccagaggcgcacccgcctccggctggatggggctcgagaggcagccggccccgagccccctcccgaggcccagccctcaccacccccaccgcctggctgctggggctgctggcagaaggggcacggggaggccatgccgctgcacaggtatggggtgggtgaggcggccgtaggcgctgaggccggggaggcgaccctcgggggtggcggtgggggcggctccgtggcttcacccacctccagctccggcagctcctcgaggggcactgagaggccgcgctcactcaagaggggctccaagccgtcggcgtcctcggcctcgctggagaagcgcatgaagatggtgtcccagagcttcacccagcgctttcggctgtctcgggacaggaaagtggccaagtcgctggccgtcatcgtgagcatctttgggctctgctgggccccatacacgctgctgatgatcatccgggccgcctgccatggccactgcgtccctgactactggtacgaaacctccttctggctcctgtgggccaactcggctgtcaaccctgtcctctaccctctgtgccaccacagcttccgccgggccttcaccaagctgctctgcccccagaagctcaaaatccagccccacagctctctagagcactgctggaagatgggctgcaccgtgagcgccgaggacaaggcggcagccgagcgctctaagatgatcgacaagaacctgcgggaggacggcgagaaggcggcacgggaggtgaagttgcttctgttaggtgctggagaatcagggaagagcaccatcgtcaagcagatgaagatcatccacgaggatggctactcagaggaggagtgccggcagtaccgtgcggttgtctacagcaacaccatccagtctatcatggccatcgtcaaagccatgggcaacctgcagatcgactttgctgacccccagcgtgcggatgatgccaggcagctgttcgcactgtcctgtgctgccgaggagcaaggcatgcttccggaagacctgtcgggcgtcatccggaggctctgggctgaccatggtgtgcaagcctgctttggccgctcacgggaatatcaactcaatgactcagccgcttactacctgaatgacctggagcgcatagcacagagtgactatatccctacacagcaggatgtgctgcggacccgtgtgaagaccacaggcatcgtcgaaacacacttcaccttcaaggacttacacttcaagatgtttgatgtgggtggtcagcgatctgagcggaagaagtggatccactgctttgagggtgtcacggccatcatcttctgtgtcgccttgagcgcgtacgacttggtgctggctgaggatgaggagatgaatcgcatgcatgagagcatgaagctgtttgatagcatctgcaataataagtggttcacagacacctccatcatcctcttcctcaacaagaaggacctgtttgaagagaagatcacacagagccccctgaccatctgtttccctgagtacacaggggccaacaagtatgacgaggcagccagctacatccagagcaagtttgaggacctgaataaacgcaaagacaccaaggagatctacacgcacttcacatgcgccaccgacaccaagaacgtgcagtttgtgtttgatgccgtcactgacgtcatcatcaagaacaacctgaaggactgtggcctcttctga

SEQ ID NO: 8 - protein, 800 aa, Artificial [sequence omitted]

SEQ ID NO: 9 - DNA, 2424 bp, Artificial
  human - rat chimera
atggagcgcgcgccgcccgacgggccgctgaacgcttcgggggcgctggcgggcgaggcggcggcggcgggcggggcgcgcggcttctcggcagcctggaccgcggtgctggccgcgctcatggcgctgctcatcgtggccacggtgctgggcaacgcgctggtcatgctcgccttcgtggccgactcgagcctccgcacccagaacaacttcttcctgctcaacctcgccatctccgacttcctcgtcggcgccttctgcatcccactgtatgtaccctacgtgctgacaggccgctggaccttcggccggggcctctgcaagctgtggctggtagtggactacctgctgtgcacctcctctgccttcaacatcgtgctcatcagctacgaccgcttcctgtcggtcacccgagcggtctcataccgggcccagcagggtgacacgcggcgggcagtgcggaagatgctgctggtgtgggtgctggccttcctgctgtacggaccagccatcctgagctgggagtacctgtccgggggcagctccatccccgagggccactgctatgccgagttcttctacaactggtacttcctcatcacggcttccaccctggagttctttacgcccttcctcagcgtcaccttctttaacctcagcatctacctgaacatccagaggcgcacccgcctccggctggatggggctcgagaggcagccggccccgagccccctcccgaggcccagccctcaccacccccaccgcctggctgctggggctgctggcagaaggggcacggggaggccatgccgctgcacaggtatggggtgggtgaggcggccgtaggcgctgaggccggggaggcgaccctcgggggtggcggtgggggcggctccgtggcttcacccacctccagctccggcagctcctcgaggggcactgagaggccgcgctcactcaagaggggctccaagccgtcggcgtcctcggcctcgctggagaagcgcatgaagatggtgtcccagagcttcacccagcgctttcggctgtctcgggacaggaaagtggccaagtcgctggccgtcatcgtgagcatctttgggctctgctgggccccatacacgctgctgatgatcatccgggccgcctgccatggccactgcgtccctgactactggtacgaaacctccttctggctcctgtgggccaactcggctgtcaaccctgtcctctaccctctgtgccaccacagcttccgccgggccttcaccaagctgctctgcccccagaagctcaaaatccagccccacagctctctagagcactgctggaaggcagcagcagcagcagcagcaatgggctgcaccgtgagcgccgaggacaaggcggcagccgagcgctctaagatgatcgacaagaacctgcgggaggacggcgagaaggcggcacgggaggtgaagttgcttctgttaggtgctggagaatcagggaagagcaccatcgtcaagcagatgaagatcatccacgaggatggctactcagaggaggagtgccggcagtaccgtgcggttgtctacagcaacaccatccagtctatcatggccatcgtcaaagccatgggcaacctgcagatcgactttgctgacccccagcgtgcggatgatgccaggcagctgttcgcactgtcctgtgctgccgaggagcaaggcatgcttccggaagacctgtcgggcgtcatccggaggctctgggctgaccatggtgtgcaagcctgctttggccgctcacgggaatatcaactcaatgactcagccgcttactacctgaatgacctggagcgcatagcacagagtgactatatccctacacagcaggatgtgctgcggacccgtgtgaagaccacaggcatcgtcgaaacacacttcaccttcaaggacttacacttcaagatgtttgatgtgggtggtcagcgatctgagcggaagaagtggatccactgctttgagggtgtcacggccatcatcttctgtgtcgccttgagcgcgtacgacttggtgctggctgaggatgaggagatgaatcgcatgcatgagagcatgaagctgtttgatagcatctgcaataataagtggttcacagacacctccatcatcctcttcctcaacaagaaggacctgtttgaagagaagatcacacagagccccctgaccatctgtttccctgagtacacaggggccaacaagtatgacgaggcagccagctacatccagagcaagtttgaggacctgaataaacgcaaagacaccaaggagatctacacgcacttcacatgcgccaccgacaccaagaacgtgcagtttgtgtttgatgccgtcactgacgtcatcatcaagaacaacctgaaggactgtggcctcttctga

SEQ ID NO: 10 - protein, 807 aa, Artificial
  human - rat chimera
MERAPPDGPLNASGALAGEAAAAGGARGFSAAWTAVLAALMALLIVATVLGNALVMLAFVADSSLRTQNNFFLLNLAISDFLVGAFCIPLYVPYVLTGRWTFGRGLCKLWLVVDYLLCTSSAFNIVLISYDRFLSVTRAVSYRAQQGDTRRAVRKMLLVWVLAFLLYGPAILSWEYLSGGSSIPEGHCYAEFFYNWYFLITASTLEFFTPFLSVTFFNLSIYLNIQRRTRLRLDGAREAAGPEPPPEAQPSPPPPPGCWGCWQKGHGEAMPLHRYGVGEAAVGAEAGEATLGGGGGGGSVASPTSSSGSSSRGTERPRSLKRGSKPSASSASLEKRMKMVSQSFTQRFRLSRDRKVAKSLAVIVSIFGLCWAPYTLLMIIRAACHGHCVPDYWYETSFWLLWANSAVNPVLYPLCHHSFRRAFTKLLCPQKLKIQPHSSLEHCWKAAAAAAAMGCTVSAEDKAAAERSKMIDKNLREDGEKAAREVKLLLLGAGESGKSTIVKQMKIIHEDGYSEEECRQYRAVVYSNTIQSIMAIVKAMGNLQIDFADPQRADDARQLFALSCAAEEQGMLPEDLSGVIRRLWADHGVQACFGRSREYQLNDSAAYYLNDLERIAQSDYIPTQQDVLRTRVKTTGIVETHFTFKDLHFKMFDVGGQRSERKKWIHCFEGVTAIIFCVALSAYDLVLAEDEEMNRMHESMKLFDSICNNKWFTDTSIILFLNKKDLFEEKITQSPLTICFPEYTGANKYDEAASYIQSKFEDLNKRKDTKEIYTHFTCATDTKNVQFVFDAVTDVIIKNNLKDCGLF

SEQ ID NO: 11 - DNA, 63 bp, artificial
  rat Galphai2 ala linker forward primer
ctctctagagcactgctggaaggcagcagcagcagcagcagcaatgggctgcaccgtgagcgc

(19) United States
(12) Patent Application Publication
(10) Pub. No.: US 2006/0188960 A1
(43) Pub. Date: Aug. 24, 2006

(54) Chimeric histamine H3 receptor

(76) Inventors: Stephen M. Waters, Branford, CT (US); James E. Krause, Madison, CT (US)

Correspondence Address: NEUROGEN CORPORATION, 35 NORTHEAST INDUSTRIAL ROAD, BRANFORD, CT, 06405, US

(21) Appl. No.: 11/355,711

(22) Filed: Feb. 16, 2006

Related U.S. Application Data
(60) Provisional application No. 60/654,540, filed on Feb. 18, 2005.

(51) Int. Cl. C07K 14/705; C07H 21/04; C12P 21/06

(52) U.S. Cl. 435/69.1; 435/320.1; 435/325; 536/23.5; 530/350

(57) ABSTRACT

Chimeric H3 receptor polypeptides are provided, as are polynucleotides encoding such polypeptides, and vectors and cells for recombinant expression of such polypeptides. Chimeric H3 receptor polynucleotides and polypeptides may be used, for example, to identify agents that specifically interact with H3 receptor. Such agents find use within therapies for humans and animals afflicted with conditions responsive to histamine H3 receptor modulation.

### CROSS-REFERENCE TO RELATED APPLICATIONS

This application claims priority to U.S. Provisional Application 60/654,540, filed Feb. 18, 2005, which is hereby incorporated by reference in its entirety.

### FIELD OF THE INVENTION

This invention relates generally to materials and methods useful for discovering agents for the treatment of conditions responsive to histamine H3 receptor modulation in humans and other animals. The invention is more specifically related to chimeric polypeptides comprising human histamine H3 receptor sequences and to polynucleotides encoding such polypeptides. Such polypeptides and polynucleotides may be used in the identification of agents that modulate H3 receptor activity.

### DESCRIPTION OF THE SEQUENCE LISTING

SEQ ID NO:1 human H3 receptor 5′ fragment forward primer

SEQ ID NO:2 human H3 receptor 5′ fragment reverse primer

SEQ ID NO:3 human H3 receptor 3′ fragment forward primer

SEQ ID NO:4 human H3receptor 3′ fragment reverse primer

SEQ ID NO:5 rat Gαᵢ₂ forward primer

SEQ ID NO:6 rat Gαᵢ₂ reverse primer

SEQ ID NO:7 chimeric human H3 receptor—rat Gαᵢ₂ cDNA sequence

SEQ ID NO:8 chimeric human H3 receptor—rat Gαᵢ₂ polypeptide sequence

SEQ ID NO:9 chimeric human H3 receptor—rat Gαᵢ₂ cDNA sequence

SEQ ID NO: 10 chimeric human H3 receptor—rat Gαᵢ₂ polypeptide sequence

SEQ ID NO: 11 Rat Gαi2 Ala linker forward primer

### BACKGROUND OF THE INVENTION

Hormones and neurotransmitters regulate a wide variety of biological functions, often via specific receptor proteins located on the surface of living cells. Many of these receptors carry out intracellular signaling via the activation of coupled guanosine triphosphate-binding proteins (G proteins); such receptors are collectively called G protein-coupled receptors or GPCRs. The important role of GPCRs in the regulation of cell and organ function has attracted attention to these receptors as targets for new pharmaceutical agents.

Histamine is a multifunctional chemical transmitter that signals through specific cell surface GPCRs. To date, four histamine receptor subtypes have been identified: H1, H2, H3 and H4. Histamine H3 receptor is a presynaptic GPCR that is found primarily in the central nervous system, although lower levels are also found in the peripheral nervous system. Genes encoding the H3 receptor have been reported in various organisms, including humans (see Lovenberg et al. (1999) Molecular Pharmacology 55:1101-07), and alternative splicing of this gene appears to result in multiple isoforms. The histamine H3 receptor is both an auto- and a hetero-receptor whose activation leads to a decrease in the release of neurotransmitters (including histamine, acetylcholine, norepinephrine and glutamate) from neurons in the brain, and is involved in the regulation of processes such as sleep and wakefulness, feeding and memory. In certain systems, the histamine H3 receptor may be constitutively active.

Antagonists of histamine H3 receptor increase synthesis and release of cerebral histamine and other neurotransmitters, inducing an extended wakefulness, an improvement in cognitive processes, a reduction in food intake and a normalization of vestibular reflexes. Such antagonists may find use as therapeutics for central nervous system disorders such as Alzheimer disease, schizophrenia, mood and attention alterations (including attention deficit hyperactivity disorder), memory and learning disorders, epilepsy, narcolepsy and cognitive deficits in psychiatric pathologies, as well as in the treatment and prevention of conditions such as obesity, eating disorders, vertigo, motion sickness and allergic rhinitis.

Accordingly, the histamine H3 receptor is an important target for new therapeutics for conditions responsive to H3 receptor modulation. Histamine H3 receptors (e.g., as components of membrane preparations), cells expressing such receptors and polynucleotides encoding such H3 receptors are needed to facilitate the discovery of such agents. The present invention fulfills this need, and provides further related advantages.

### SUMMARY OF THE INVENTION

The present invention provides compositions and methods for the identification of therapeutic agents useful for treating conditions responsive to histamine H3 receptor modulation. In one aspect, the present invention provides chimeric H3 receptor polypeptides, comprising an H3 receptor amino acid sequence linked contiguously with a G protein amino acid sequence, such as a G protein alpha subunit amino acid sequence (e.g., a Gαᵢ₂ amino acid sequence). In certain such polypeptides, the H3 receptor sequence is a human H3 receptor sequence and the G protein amino acid sequence is a rat Gαᵢ₂ sequence. In one embodiment, the chimeric H3 receptor polypeptide comprises the amino acid sequence recited in SEQ ID NO:8. In another embodiment, the chimeric H3 receptor polypeptide comprises the amino acid sequence recited in SEQ ID NO:10.

In another aspect, the present invention provides polynucleotides (which may be DNA, such as cDNA, or RNA) that encode a chimeric H3 receptor polypeptide as described above. In one such embodiment, the polynucleotide comprises the sequence recited in SEQ ID NO:7. In another such embodiment, the polynucleotide comprises the sequence recited in SEQ ID NO:9.

Within further aspects, nucleic acid expression vectors are provided, comprising a promoter and a polynucleotide as described above, wherein the polynucleotide is functionally linked to the promoter so that a chimeric H3 receptor polypeptide will be expressed upon introduction of the expression vector into a cell of at least one cell type. Such vectors include, for example, expression cassettes, plasmids and viral vectors. Also provided are recombinant cells that comprise an expression vector as described above, wherein the cell is of a cell type that will express a chimeric polypeptide encoded by the expression vector, and chimeric H3 receptor polypeptide-comprising cell membrane preparations isolated from such cells. In certain embodiments, the cell membrane preparation exhibits H3 receptor agonist-stimulated GTP binding activity that is at least 2-fold greater than H3 receptor agonist-stimulated GTP binding activity exhibited by a control membrane preparation isolated from cells of the same cell type that do not contain an expression vector comprising a polynucleotide encoding a chimeric H3 receptor polypeptide.

Methods are provided, within other aspects, for determining H3 receptor modulating activity of a compound. Such methods generally comprise the steps of: (a) contacting a compound with a cell membrane preparation as described above (optionally in the presence of H3 receptor agonist); and (b) detecting an effect of the compound on H3 receptor GTP binding activity of the cell membrane preparation. Certain such methods comprise the steps of:

(a) simultaneously or in either order:
i) contacting a cell membrane preparation as described above with a fixed concentration of H3 receptor agonist, wherein the fixed concentration is sufficient to stimulate GTP binding activity of the chimeric H3 receptor polypeptide, and thereby generating a control agonist-stimulated sample;
ii) contacting a cell membrane preparation as described above with the fixed concentration of H3 receptor agonist and a test compound, and thereby generating a test agonist-stimulated sample;

(b) detecting a response indicative of chimeric H3 receptor GTP binding activity in the control agonist-stimulated sample and in the test agonist-stimulated sample; and

(c) comparing the response detected in the test agonist-stimulated sample with the response detected in the control agonist-stimulated sample, and therefrom determining H3 receptor modulating activity of the compound.

These and other aspects of the present invention will become apparent upon reference to the following detailed description.

### DETAILED DESCRIPTION

As noted above, the present invention is generally directed to compounds and methods for identifying therapeutic agents useful for treating conditions responsive to histamine H3 receptor modulation. Compounds provided herein include chimeric H3 receptor polypeptides, which comprise a H3 receptor amino acid sequence and a G protein amino acid sequence, as well as polynucleotides encoding such polypeptides. Chimeric H3 receptor polypeptides and polynucleotides encoding such polypeptides may be used to identify therapeutic agents that are useful for H3 receptor activity in a variety of contexts.

Chimeric H3 Receptor Polypeptides

As used herein, the phrase “chimeric H3 receptor polypeptide” (or “CHP”) refers to a single polypeptide that comprises both an H3 receptor amino acid sequence and a G protein amino acid sequence, provided that the chimeric polypeptide retains H3 receptor function, as determined using the assay provided in Example 4, herein. The fusion of H3 receptor sequence to G protein sequence may, in certain embodiments, enhance receptor activity in functional assays (i.e., the chimeric receptor may be more active in such assays than the corresponding unmodified H3 receptor), thereby improving signal within such assays and/or decreasing the amount of exogenous G protein (e.g., generated by co-transfection in recombinant cells) required for optimal signal in such assays. Preferably the H3 receptor amino acid sequence is located on the N-terminal portion of the polypeptide. Contiguous linkage between the H3 receptor amino acid sequence and the G protein amino acid sequence may be with or without intervening sequence such as a short, contiguous linker peptide.

The H3 receptor amino acid sequence is the amino acid sequence of any histamine H3 subtype receptor, including human H3 receptor (see, e.g., U.S. Pat. No. 6,136,559) and H3 receptor from other mammals. The H3 receptor amino acid sequence may be a sequence of a naturally-occurring H3 receptor, or such a sequence may be modified, provided that any modifications do not substantially diminish receptor function, as determined using the assay provided in Example 4, herein (i.e., the agonist-stimulated GTP binding activity is enhanced, unchanged or diminished by no more than 10%). Such modifications may include amino acid deletions, insertions and/or substitutions, and may occur at any point in the naturally-occurring sequence. A “human CHP” is a CHP in which the H3 receptor amino acid sequence is a human sequence, regardless of the origin of the G protein sequence.

The G protein amino acid sequence is the amino acid sequence of any monomeric G protein or heterotrimeric G protein subunit. In certain embodiments, the G protein amino acid sequence is a G protein alpha subunit sequence, preferably a Gαᵢ isoform (e.g., a Gαᵢ₂ amino acid sequence), although other isoforms may also be used (e.g., Gαᵢ₅, Gαᵢ₆, Gα_{q} or Gαₛ). The G protein sequence may be a naturally-occurring sequence (e.g., human or rat), or may be a modified sequence (e.g., a G protein alpha subunit sequence that is mutated to change coupling selectivity, or that is itself a chimeric sequence comprising sequences derived from multiple G protein alpha subunit isoforms).

Representative CHPs include polypeptides comprising the sequences recited in SEQ ID NOs:8 and 10.

CHPs may be prepared using standard recombinant methods. For example, convenient restriction sites may be found or incorporated into polynucleotides encoding portions of the CHP. The portions may, for example, be isolated from plasmids, synthesized or PCR amplified, and ligated to generate a polynucleotide encoding the CHP. CHPs may then be prepared using any of a variety of well known techniques from recombinant cells (i.e., cells that have been genetically altered to express a CHP). Recombinant polypeptides encoded by nucleic acid sequences as described above may be readily prepared by methods well known in the art. Expression may be achieved in any appropriate host cell that has been transformed, transfected, microinjected, or otherwise prepared so as to comprise at least one expression vector containing a nucleic acid sequence that encodes a CHP. Suitable host cells include prokaryotic, yeast and higher eukaryotic cells, such as insect, mammalian or plant cells. For example, E. coli, yeast, amphibian oocytes, insect cell lines such as SJ9 cells, and mammalian cell lines such as COS, CHO, BHK, HEK 293, VERO, HeLa, MDCK, W138 or NIH 3T3 cells may be used. Insect cell systems may be prepared by infection with recombinant viral expression vectors (for example, baculovirus) comprising a CHP-encoding polynucleotide provided herein. Alternatively, mammalian cell lines may be transiently or stably transfected, or a transgenic cell may be isolated from a transgenic animal.

In certain embodiments, CHPs are purified. A polypeptide is said to be “purified” if it represents at least 1% of total polypeptide molecules (preferably at least 10% and more preferably at least 20%) of total polypeptide molecules, within a sample or preparation. Similarly, a CHP-encoding polynucleotide is purified if it represents at least 1% of total nucleic acid molecules (preferably at least 10% and more preferably at least 20%) of total nucleic acid molecules, within a sample or preparation.

CHPs may also be isolated as membrane preparations. Such preparations are generated from recombinant cells that express a CHP, using any standard procedure. For example, transfected host cell pellets may be homogenized and centrifuged. The supernatant is discarded and the pellet is resuspended and homogenized again to generate an isolated membrane preparation. A more detailed protocol is provided in Example 3, herein. Preferably, isolated membranes exhibit H3 receptor activity that is at least 2-fold greater, preferably 10-fold greater and more preferably at least 20-fold greater than that exhibited by control membranes isolated from a control cell (e.g., an untransfected cell of the same cell line used to prepare the recombinant cell or a cell transfected with a control vector that does not encode a CHP). Preferred membranes contain at least 0.1 pmol, 1 pmol or 5 pmol of CHP per mg of total membrane protein.

Chimeric H3 Receptor Polynucleotides

Any polynucleotide that encodes a CHP as described herein is encompassed by the present invention. Polynucleotides may be single-stranded (coding or antisense) or double-stranded, and may be DNA (genomic, cDNA or synthetic) or RNA, such as mRNA molecules. Additional coding or non-coding sequences may, but need not, be present within a polynucleotide of the present invention, and a polynucleotide may, but need not, be linked to other molecules and/or support materials.

Certain CHP-encoding polynucleotides encode a human CHP, such as the polypeptide provided in SEQ ID NO:8. One such polynucleotide comprises the sequence provided in SEQ ID NO:7. Another CHP-encoding polynucleotide, provided herein comprises the sequence recited in SEQ ID NO:9, which encodes a CHP having the sequence recited in SEQ ID NO:10. It will be appreciated by those of ordinary skill in the art that, as a result of the degeneracy of the genetic code, there are many nucleotide sequences that encode each specific CHP. Some of these polynucleotides bear reduced homology to the nucleotide sequence of any naturally occurring gene. Nonetheless, polynucleotides that vary due to differences in codon usage are specifically contemplated by the present invention. Additionally, it will be apparent that sequence changes may be made in the non-coding regions (if any) of the polynucleotides without altering the amino acid sequence of the protein product. Polynucleotides that encode amino acid sequences with up to 15 (preferably no more than 10, more preferably no more than 5) amino acid substitutions relative to a native sequence are also provided herein.

Polynucleotides provided herein may further comprise additional sequences. For example, an optimized translation initiation sequence (Kozak sequence) may be added 5′ to the coding region. In-frame additions of sequences encoding antibody recognition sites may also be included as in-frame inserts. Such amino acid sequences include, but are not limited to, the His-6× (hexa-histidine) epitope which is specifically bound by the Monoclonal Anti-polyhistidine Clone HIS-1 monoclonal antibody (Sigma, St. Louis No.H1029), and the FLAG epitope which is specifically bound by the FLAG-M2 monoclonal antibody (Sigma, St. Louis No. F3165). Such modifications may be readily introduced using routine methods or by using prepared kits, such as the Sigma Mammalian FLAG Expression Kits (Sigma, St. Louis). Preferably, fusions are made as amino- (N-) or carboxy- (C-) terminal fusions or as linkers joining the H3 receptor and G protein moieties of the CHP. Unless otherwise specified, a polynucleotide comprising a given sequence may be of any length sufficient to comprise the sequence.

Polynucleotides may be prepared using any of a variety of well known techniques. For example, polynucleotides (or portions thereof) may be amplified via polymerase chain reaction (PCR), using sequence-specific primers designed based on the sequences provided herein, which may be purchased or synthesized. Portions of a desired polynucleotide obtained using PCR may be assembled into a single contiguous sequence by ligating suitable fragments, using well known techniques.

Polynucleotides containing nucleotide substitutions, additions and/or deletions may generally be prepared by any standard method, including chemical synthesis by, for example, solid phase phosphoramidite chemical synthesis. Modifications in a polynucleotide sequence may also be introduced using standard mutagenesis techniques, such as oligonucleotide-directed site-directed mutagenesis.

Polynucleotides as described herein may be joined to a variety of other polynucleotides using established recombinant DNA techniques. For example, a polynucleotide may be cloned into any of a variety of cloning vectors, including expression cassettes, plasmids, phagemids, lambda phage derivatives and cosmids. Vectors of particular interest include expression vectors, replication vectors, probe generation vectors and sequencing vectors. In general, a vector will contain an origin of replication that is functional in at least one organism, convenient restriction endonuclease sites and one or more selectable markers. Other elements will depend upon the desired use, and will be apparent to those of ordinary skill in the art.

Expression Systems

An expression vector is a vector for recombinant expression of a CHP, and comprises a CHP-encoding polynucleotide operatively linked to the necessary nucleotide sequences for expression (e.g., a suitable promoter and, if necessary, a terminating signal). A promoter is a nucleotide sequence (typically located 5′ to the CHP-encoding polynucleotide) that directs the transcription of adjacently linked coding sequences. A terminating signal may be a stop codon to end translation and/or a transcription termination signal. Additional regulatory element(s) (e.g., enhancer elements) may also be present within an expression vector. Such a vector is preferably a plasmid or viral vector. Techniques for incorporating DNA into such vectors are well known to those of ordinary skill in the art.

Preferably, an expression vector further comprises a selectable marker, which confers resistance to a selection. This allows cells to stably integrate the vector into their chromosomes and grow to form foci, which in turn can be cloned and expanded into cell lines. A number of selection systems can be used. For example, the hypoxanthine-guanine phosphoribosyl-transferase, adenine phosphoribosyltransferase and herpes simplex virus thymidine kinase genes can be employed in hgprt⁻, aprt or tk⁻ cells, respectively. Also, anti-metabolite resistance can be used as the basis of selection for genes such as: dhfr, which confers resistance to methotrexate; gpt, which confers resistance to mycophenolic acid; neo, which confers resistance to the aminoglycoside G-418; hygro, which confers resistance to hygromycin; and puro, which confers resistance to puromycin.

Expression systems that may be used in the practice of certain aspects of the present invention include, but are not limited to, (a) insect cell systems infected with recombinant virus expression vectors (e.g., baculovirus) comprising one or more polynucleotides provided herein and (b) mammalian cell systems (e.g., COS, CHO, BHK, HEK 293, VERO, HeLa, MDCK, W138 and NIH 3T3 cells) harboring recombinant expression constructs comprising one or more polynucleotides provided herein.

Mammalian vectors should contain promoters, preferably derived from the genome of a mammalian cell (for example, a metallothionein actin or phosphoglycerate kinase promoter) or from mammalian viruses (for example, the adenovirus late promoter, a CMV promoter and the vaccinia virus 7.5K promoter). In adenoviral expression vectors, the CHP-encoding polynucleotide may be ligated to an adenovirus transcription/translation control complex such as the late promoter and tripartite leader sequence. Specific initiation signals (e.g., the ATG initiation codon and adjacent sequences such as ribosome binding sites) may also be required for efficient translation of inserted nucleic acid molecules. The efficiency of expression may be further enhanced by the inclusion of appropriate transcription enhancer elements, transcription terminators, etc. The recombinant expression cassette may then be inserted in the adenovirus genome by in vitro or in vivo recombination. Insertion in a non-essential region of the viral genome (for example, region E1 or E3) will result in a recombinant virus that is viable and capable of expressing a CHP in infected cells.

Another representative expression system is an amphibian oocyte system in which a CHP-encoding RNA is introduced into an oocyte. Preferably the amphibian is a frog, most preferably the African clawed frog, Xenopus laveis. One suitable expression vector for expression in amphibian oocytes is the pBLUESCRIPT SK⁻ vector (STRATAGENE, La Jolla, Calif.). Typically such vectors are used to generate CHP-encoding RNAs in in vitro transcription systems, which RNAs are then injected into the oocytes to induce expression of the encoded protein.

An insect cell system utilizing a baculovirus such as Autographa californica nuclear polyhedrosis virus (AcNPV) can be used to express the CHPs provided herein. The virus grows in insect cells such as Spodoptera frugiperda cells. The coding sequence encoding the CHP is typically inserted (e.g., ligated) into non-essential regions of the virus (for example into the polyhedrin gene) and placed under control of an AcNPV promoter (for example the polyhedrin promoter). Preferably, the successful introduction of the insert will result in inactivation of a viral gene. For example, when targeted into the polyhedrin gene, the successful incorporation of the insert will inactivate that gene and result in production of non-occluded recombinant virus (i.e., virus lacking the proteinaceous coat coded for by the polyhedrin gene). The resulting recombinant viruses are then used to infect insect cells, preferably Spodoptera frugiperda cells, in which the inserted coding sequence is expressed. A variety of kits for use in the preparation of an insect expression system are commercially available.

Host cells transformed or transfected with an expression vector comprising a CHP-encoding polynucleotide, and capable of expressing a CHP, are further provided herein. Such cells may be prepared using standard transformation techniques. Stable expression is generally preferred, although transient expression systems may be suitable for certain uses. Following the introduction of the vector (often following incubation in a non-selective medium to allow for recovery from the stress of vector introduction), engineered cells may be grown in a selective medium. Also provided are transgenic animals (preferably rodents such as mice or rats) engineered to express a CHP.

Assays

CHP-encoding polynucleotides and CHPs may be used within a variety of assays to screen for and characterize compounds that modulate H3 receptor function. Such assays typically involve contacting a compound with a CHP preparation (such as cells that recombinantly express CHP or membrane preparations prepared from such cells as described above), and subsequently detecting (a) binding of the compound to the cells or membranes (direct binding assays—e.g., via surface plasmon resonance, using a device available from BIAcor AB, Sweden); (b) an effect of the compound on labeled ligand (e.g., radiolabeled ligand) binding to the cells or membranes (competitive binding assays); or (c) an effect on a cellular receptor response to H3 receptor activity (functional assays). Compounds may be any substance, but are preferably small organic, non-peptide molecules. Compounds identified using such assays are useful, for example, as tools for receptor mapping and as pharmaceutical agents.

Certain functional assays employ cell membrane preparations that are generated from recombinant cells that express a CHP, as described above. In general, such a membrane preparation is contacted with a test compound, and the effect of contact with test compound on H3 receptor GTP binding activity of the cell membrane preparation is determined. Within assays for H3 receptor agonist-stimulated GTP binding activity, such a membrane preparation is contacted with a test compound in the presence of H3 receptor agonist (e.g., histamine or an analogue thereof such as R-alpha-methylhistamine), and the effect of contact with test compound on H3 receptor agonist-stimulated GTP binding activity of the cell membrane preparation is determined. Within assays for H3 receptor agonist or inverse agonist activity, such a membrane preparation is contacted with a test compound in the absence of added H3 receptor agonist, and the effect of contact with test compound on H3 receptor GTP binding activity of the cell membrane preparation is determined.

Commonly, the effect of test compound on H3 receptor GTP binding activity is determined by comparing a signal that is indicative of H3 receptor agonist-stimulated GTP binding activity in test cells (cells contacted with test compound) with a signal that is indicative of H3 receptor agonist-stimulated GTP binding activity in control cells (cells treated similarly to test cells, except for the absence of test compound). It will be apparent that the incubation conditions should be the same in the test and control samples, such that both samples are substantially the same except for the presence of test compound in the test sample. Similarly, if agonist is employed, the concentration of agonist used should be the same in control and test samples, and should be sufficient to detectably stimulate GTP binding activity of the chimeric H3 receptor polypeptide in control cells under the conditions employed. The response indicative of H3 receptor agonist-stimulated GTP binding activity may be, for example, membrane-bound radioactivity, if radiolabeled GTP is employed in the assay. Membrane-bound radiolabeled GTP can be conveniently detected using liquid scintillation counting. The effect of the test compound on H3 receptor agonist-stimulated GTP binding activity is then determined by comparing the membrane-bound radiolabeled GTP in the test cells with the membrane-bound radiolabeled GTP in the control cells.

It will be apparent that such assays may be performed with varying concentrations of agonist and/or varying concentrations of test compound in order to further characterize the activity of the test compound. Alternatively, to assess inverse agonist or agonist activity of a test compound, the GTP binding assay may be performed in the absence of added agonist. For example, a comparison may be made between cells contacted with test compound in the absence of added agonist and cells contacted with neither exogenous agonist nor test compound. Neutral antagonists are those test compounds that reduce agonist-stimulated GTP binding activity towards, but not below, baseline levels. In contrast, in the absence of added agonist, inverse agonists reduce the GTP binding activity of the receptor-containing membranes below baseline. Any test compound that elevates GTP binding activity above baseline in the absence of added agonist in this assay is defined as having agonist activity. An assay to more completely characterize activity of a test compound may be performed, for example, by incubating four separate equivalent aliquots of a cell membrane preparation as described above: i) with a fixed concentration of H3 receptor agonist, wherein the fixed concentration is sufficient to stimulate GTP binding activity of the chimeric H3 receptor polypeptide (a control agonist-stimulated sample); ii) with the fixed concentration of H3 receptor agonist and a fixed concentration of test compound (a test agonist-stimulated sample); iii) in the absence of exogenous H3 receptor agonist or test compound (a control sample); and iv) with the fixed concentration of test compound (a test sample). Following incubation, a response indicative of chimeric H3 receptor agonist-stimulated GTP binding activity is detected in the test agonist-stimulated sample and control agonist-stimulated sample; and the two responses are compared. Similarly, a response indicative of chimeric H3 receptor GTP binding activity in the absence of agonist is detected in the control sample and the test sample; and those two responses are compared. Such an assay permits the detection of antagonist, inverse agonist and agonist activity.

Example 4 provides a representative H3 receptor GTP binding assay, which may be performed as an H3 receptor agonist-stimulated GTP binding assay or may be performed in the absence of added agonist. Briefly, within a H3 receptor agonist-stimulated GTP binding assay, a membrane preparation from cells that recombinantly express a CHP is incubated with a H3 receptor agonist (e.g., histamine), labeled (e.g., ³⁵S) GTP and unlabeled test compound. Incubation with a compound that modulates H3 receptor function results in a decrease or increase in the amount of label bound to the CHP preparation, relative to the amount of label bound in the absence of the compound. A series of varying concentrations may be used to generate dose response curves, from which IC₅₀ and Kᵢ values may be determined using standard techniques, and as described in Example 4. Preferably, such a compound will exhibit a Kᵢ receptor of less than 1 micromolar, more preferably less than 500 nM, 100 nM, 20 nM or 10 nM, within an assay performed as described in Example 4.

Compounds identified using such assays may be used to treat one or more conditions responsive to histamine H3 receptor modulation, such as attention deficit disorder, attention deficit hyperactivity disorder, schizophrenia, cognitive disorders (including mild cognitive impairment), epilepsy, migraine, narcolepsy, allergic rhinitis, vertigo, motion sickness, memory disorders such as Alzheimer's disease, Parkinson's disease, obesity, an eating disorder or diabetes. Patients may include humans, companion animals (such as dogs) and livestock animals.

The following Examples are offered by way of illustration and not by way of limitation.

### EXAMPLES

### Example 1

### Preparation of Representative CHP-Encoding Polynucleotide

PCR cloning is used to prepare a CHP-encoding cDNA from human H3 receptor cDNA directly coupled to a rat Gαᵢ₂ cDNA. The final construct is generated from three cDNA fragments: (1) a human H3 receptor cDNA 5′ fragment; (2) a human H3 receptor cDNA 3′ fragment; and (3) a rat Gαᵢ₂ cDNA fragment, each containing appropriate, overlapping linker sequences.

The human H3 receptor cDNA 5′ fragment is generated from a Human Brain cDNA Library (Invitrogen, Carlsbad, Calif.) by PCR using the primers TGAGCCTGCGGGGCCATGGAG (forward, SEQ ID NO:1) and GAGGAGGTGCACAGCAGGTAG (reverse, SEQ ID NO:2). PCR is performed using the Advantage-GC cDNA PCR kit (BD Biosciences Clontech, Palo Alto, Calif.) in 25 μl reactions containing: 5 μl GC melt, 5 μl 5× PCR reaction buffer, 0.5 μl dNTP Mix (10 mM each), 10 pmol forward and reverse primers, 0.5 μl Advantage GC cDNA Polymerase Mix and 1 μl undiluted Invitrogen Human Brain Library. Conditions for touchdown PCR thermal cycling are 94° C. for 2 minutes then, over 10 cycles: 94° C. for 30 seconds, 60° C. to 55° C. intervals for 1 minute and 68° C. for 1 minute, then over 20 cycles: 94° C. for 30 seconds, 55° C. for 1 minute and 68° C. for 1 minute. The human H3 receptor cDNA 5′ fragment is initially cloned into the vector pcDNA3.1/V5His TOPO TA (Invitrogen).

The human H3 receptor cDNA 3′ fragment is generated from a Human Brain Library (Invitrogen) by PCR using the primers CTACCTGCTGTGCACCTCCTC (forward, SEQ ID NO:3) and GCAGTGCTCTAGAGAGCTGTGG (reverse, SEQ ID NO:4) and conditions as described for the human H3 receptor cDNA 5′ fragment. The human H3 receptor cDNA 3′ fragment is initially cloned into the vector pcDNA3.1/V5His TOPO TA (Invitrogen).

The rat Gαᵢ₂ cDNA fragment is generated from a rat Gaᵢ₂-containing vector (Jones and Reed (1987) J. Biol. Chem. 262(29): 14241-49) by PCR using the primers CTCTCTAGAGCACTGCTGGAAGATGGGCTGCACCGTGAGCGC (forward, SEQ ID NO:5) and TCAGAAGAGGCCACAGTCCTTCAG (reverse, SEQ ID NO:6) and conditions as described for the human H3 receptor cDNA 5′ fragment. The rat Gαᵢ₂ cDNA fragment is initially cloned into the vector pcDNA3.1/V5His TOPO TA (Invitrogen).

Each cDNA fragment is excised from the pcDNA3.1/V5His TOPO TA vector using the restriction enzymes KpnI and NotI. The human H3 receptor cDNA 5′ fragment is further treated with the restriction enzyme ApaLI. The human H3 receptor cDNA 3′ fragment is further treated with the restriction enzymes ApaLI and XbaI. The rat Gαᵢ₂ cDNA fragment is further treated with the restriction enzyme XbaI. All three individual cDNA fragments are ligated and subcloned together into the KpnI/NotI site of the baculoviral expression vector pBacPAK 9 (BD Biosciences Clontech) to generate the CHP baculoviral expression construct (SEQ ID NO:7).

To prepare a CHP that comprises an Ala linker, a rat Gαᵢ₂ cDNA fragment containing a 5′ Ala linker is generated from a rat Gαᵢ₂-containing vector as described above, except that the forward primer is CTCTCTAGAGCACTGCTGGAAGGCAGCAGCAGCAGCAGCAGCAATGGGCTG CACCGTGAGCGC (SEQ ID NO:11). The human H3 receptor cDNA 5′ and 3′ fragments are generated as described above and all three fragments are initially cloned into the vector pcDNA3.1/V5His TOPO TA (Invitrogen) as described above. Each cDNA fragment is excised from the vector using the restriction enzymes KpnI and NotI. The human H3 receptor cDNA 5′ fragment is further treated with the restriction enzyme ApaLI. The human H3 receptor cDNA 3′ fragment is further treated with the restriction enzymes ApaLI and XbaI. The rat Gαᵢ₂ cDNA fragment is further treated with the restriction enzyme XbaI. All three individual cDNA fragments are ligated and subcloned together into the KpnI/NotI site of the baculoviral expression vector pBacPAK 9 (BD Biosciences Clontech) to generate the CHP baculoviral expression construct (SEQ ID NO:9).

### Example 2

### CHP Baculovirus Preparation and Infection

The CHP baculoviral expression vector of Example 1 is co-transfected along with BACULOGOLD DNA (BD PharMingen, San Diego, Calif.) into Sf9 cells. The Sf9 cell culture supernatant is harvested three days post-transfection. The recombinant virus-containing supernatant is serially diluted in Hink's TNM-FH insect medium (JRH Biosciences, Kansas City, Kans.) supplemented Grace's salts and with 4.1 mM L-Gln, 3.3 g/L LAH, 3.3 g/L ultrafiltered yeastolate and 10% heat-inactivated fetal bovine serum (hereinafter “insect medium”) and plaque assayed for recombinant plaques. After four days, recombinant plaques are selected and harvested into 1 ml of insect medium for amplification. Each 1 ml volume of recombinant baculovirus (at passage 0) is used to infect a separate T25 flask containing 2×10⁶ Sf9 cells in 5 ml of insect medium. After five days of incubation at 27° C., supernatant medium is harvested from each of the T25 infections for use as passage 1 inoculum.

Two of seven recombinant baculoviral clones are chosen for a second round of amplification, using 1 ml of passage 1 stock to infect 1×10⁸ cells in 100 ml of insect medium divided into two T175 flasks. Forty-eight hours post infection, passage 2 medium from each 100 ml prep is harvested and plaque assayed to determine virus titer. The cell pellets from the second round of amplification are assayed by affinity binding as described below to verify recombinant receptor expression. A third round of amplification is then initiated using a multiplicity of infection of 0.1 to infect a liter of Sj9 cells. Forty hours post-infection, the supernatant medium is harvested to yield passage 3 baculoviral stock.

The remaining cell pellet is assayed for affinity binding to histamine receptor ligand using the protocol of DeMartino et al. (1994) J. Biol. Chem. 269(20):14446-50 (which is incorporated herein by reference for its teaching of binding assays at page 14447), adapted as follows. Radioligand ranges from 0.40-40 nM [³H]-N-(a)methylhistamine (Perkin Elmer, Boston, Mass.) and assay buffer contains 50 mM Tris, 1 mM CaCl₂, 5 mM MgCl₂, 0.1% BSA, 0.1 mM bacitracin, and 100 KIU/ml aprotinin, pH 7.4. Filtration is carried out using GF/C WHATMAN filters (presoaked in 1.0% polyethyeneimine for 2 hours prior to use). Filters are washed three times with 5 ml cold binding buffer without BSA, bacitracin, or aprotinin and air dried for 12-16 hours. Radioactivity retained on filters is measured on a beta scintillation counter.

Titer of the passage 3 baculoviral stock is determined by plaque assay and a multiplicity of infection, incubation time course, binding assay experiment is carried out to determine conditions for optimal receptor expression. A multiplicity of infection of 0.5 and a 72-hour incubation period were the best infection parameters found for expression in up to 1-liter SJ9 cell infection cultures.

Log-phase SJ9 cells (Invitrogen), are infected with one or more stocks of recombinant baculovirus followed by culturing in insect medium at 27° C. Infections are carried out with virus directing the expression of human H3 receptor-rat Gαᵢ₂ in combination with three G-protein subunit-expression virus stocks: 1) rat Gαᵢ₂ G-protein-encoding virus stock (BIOSIGNAL #V5J008), 2) bovine β1 G-protein-encoding virus stock (BIOSIGNAL #V5H012), and 3) human γ2 G-protein-encoding virus stock (BIOSIGNAL #V6B003), which may be obtained from BIOSIGNAL Inc., Montreal.

The infections are conveniently carried out at a multiplicity of infection of 0.5:1.0:0.5:0.5. At 72 hours post-infection, a sample of cell suspension is analyzed for viability by trypan blue dye exclusion, and the remaining cells are harvested as Sf9 pellets via centrifugation (3000 rpm/10 minutes/4° C.).

### Example 3

### CHP Cell Membrane Preparations

Sf9 cell pellets of Example 2 are resuspended in homogenization buffer (10 mM HEPES, 250 mM sucrose, 0.5 μg/ml leupeptin, 2 μg/ml Aprotinin, 200 μM PMSF, and 2.5 mM EDTA, pH 7.4) and homogenized using a POLYTRON homogenizer (setting 5 for 30 seconds). The homogenate is centrifuged (536×g/10 minutes at 4° C.) to pellet the nuclei and unbroken cells. The supernatant containing the membranes is decanted to a clean centrifuge tube, centrifuged (48,000×g/30 minutes, 4° C.) and the resulting pellet resuspended in 30 ml homogenization buffer. This centrifugation and resuspension step is repeated twice. The final pellet is resuspended in ice cold Dulbecco's PBS containing 5 mM EDTA and stored in frozen aliquots at −80° C. until used for radioligand binding or functional response assays. The protein concentration of the resulting membrane preparation (hereinafter termed “P2 membranes”) is conveniently measured using a Bradford protein assay (Bio-Rad Laboratories, Hercules, Calif.). By this measure, a 1-liter culture of cells typically yields 100-150 mg of total membrane protein.

### Example 4

### CHP GTP Binding Assays

Agonist-stimulated GTP-gamma³⁵S binding (“GTP binding”) activity can be used to assess activity of a CHP and to identify compounds that antagonize such activity. A compound being analyzed in this assay is referred to herein as a “test compound.” Agonist-stimulated GTP binding activity is measured as follows: Four independent baculoviral stocks (one directing the expression of the CHP and three directing the expression of each of the three subunits of a heterotrimeric G-protein) are used to infect a culture of Sj9 cells as described above.

Agonist-stimulated GTP binding on P2 membranes of Example 3 is assessed using histamine (Sigma Chemical Co., St. Louis, Mo.) as agonist in order to ascertain that the receptor/G-protein-alpha-beta-gamma combination(s) yield a functional response as measured by GTP binding.

P2 membranes are resuspended by Dounce homogenization (tight pestle) in GTP binding assay buffer (50 mM Tris pH 7.0, 120 mM NaCl, 2 mM MgCl₂, 2 mM EGTA, 0.1% BSA, 0.1 mM bacitracin, 100KIU/mi aprotinin, 10 μM GDP) and added to assay tubes at a concentration of 35 μg protein/reaction tube. After adding increasing doses of histamine at concentrations ranging from 10⁻¹² M to 10⁻⁵ M, reactions are initiated by the addition of 125 pM GTP-gamma³⁵S with a final assay volume of 0.20 ml. In competition experiments, non-radiolabeled test compounds are added to separate reactions at concentrations ranging from 10⁻¹⁰ M to 10⁻⁶ M along with 1 μM histamine to yield a final volume of 0.20 ml.

After a 60-minute incubation at room temperature, reactions are terminated by vacuum filtration over GF/C filters (pre-soaked in wash buffer, 0.1% BSA) followed by washing with ice-cold wash buffer (50 mM Tris pH 7.4, 120 mM NaCl). The amount of receptor-bound (and thereby membrane-bound) GTP-gamma³⁵S is determined by measuring the bound radioactivity, preferably by liquid scintillation spectrometry of the washed filters. Non-specific binding is determined using 10 pM GTP-gammaS and typically represents less than 5 percent of total binding. Data is expressed as percent above basal (baseline). The results of GTP binding experiments are analyzed using SIGMAPLOT software (SPSS Inc., Chicago, Ill.).

IC₅₀ values are calculated by non-linear regression analysis of dose-response curves using Kaleidograph (Synergy Software, Reading, Pa.). Calculated IC₅₀ values are converted to Kᵢ values by the Cheng-Prusoff correction (Cheng and Prusoff (1973) Biochem. Pharmacol. 22(23):3099-3108). Accordingly, the following equation: Kᵢ=IC₅₀/(1+[L]/EC₅₀) is used, where [L] is the histamine concentration in the GTP binding assay, and EC₅₀ is the concentration of histamine producing a 50% response, as determined by a dose-response analysis using concentrations of histamine ranging from 10⁻¹⁰ M to 10⁻⁶M.

To assess agonist or inverse agonist activity of a test compound, this assay is performed in the absence of added histamine.

1. A chimeric H3 receptor polypeptide, comprising an H3 receptor amino acid sequence and a G protein amino acid sequence.

2-5. (canceled)

6. A polynucleotide encoding a chimeric H3 receptor polypeptide according to claim 1.

7. A polynucleotide according to claim 6, wherein the polynucleotide encodes a polypeptide that comprises the amino acid sequence recited in SEQ ID NO:8.

8. A polynucleotide according to claim 7, wherein the polynucleotide comprises the sequence recited in SEQ ID NO:7.

9. A polynucleotide according to claim 6, wherein the polynucleotide encodes a polypeptide that comprises the amino acid sequence recited in SEQ ID NO:10.

10. A polynucleotide according to claim 9, wherein the polynucleotide comprises the sequence recited in SEQ ID NO:9.

11. An expression vector comprising a promoter and a polynucleotide according to claim 6, wherein the polynucleotide is functionally linked to the promoter so that a chimeric H3 receptor polypeptide will be expressed upon introduction of the expression vector into a cell of at least one cell type.

12. An expression vector according to claim 11, wherein the vector is a plasmid.

13. An expression vector according to claim 11, wherein the vector is a viral vector.

14. A recombinant cell comprising an expression vector according to claim 11, wherein the cell is of a cell type that will express a chimeric H3 receptor polypeptide encoded by the expression vector.

15. A recombinant cell according to claim 14, wherein the cell is a mammalian cell.

16. A recombinant cell according to claim 14, wherein the cell is an oocyte.

17. A recombinant cell according to claim 14, wherein the cell is an insect cell.

18. A chimeric H3 receptor polypeptide-comprising cell membrane preparation isolated from a recombinant cell according to claim 14.

19. A chimeric H3 receptor polypeptide-comprising cell membrane preparation according to claim 18, wherein the cell membrane preparation exhibits H3 receptor agonist-stimulated GTP binding activity that is at least 2-fold greater than H3 receptor agonist-stimulated GTP binding activity exhibited by a control membrane preparation isolated from cells of the same cell type that do not contain an expression vector comprising a polynucleotide encoding a chimeric H3 receptor polypeptide.

20. A method for determining H3 receptor modulating activity of a compound, comprising the steps of:
(a) contacting a compound with a cell membrane preparation according to claim 18; and
(b) detecting an effect of the compound on H3 receptor GTP binding activity of the cell membrane preparation, and therefrom determining H3 receptor modulating activity of the compound.

21. A method according to claim 20, comprising the steps of:
(a) simultaneously or in either order:
i) incubating a cell membrane preparation according to claim 18 in the absence of test compound, and thereby generating a control sample; and
ii) contacting a cell membrane preparation according to claim 18 with a fixed concentration of test compound, to generate a test sample;
(b) detecting a response indicative of chimeric H3 receptor GTP binding activity in the control sample and in the test sample; and
(c) comparing the response detected in the test sample with the response detected in the control sample;
and therefrom determining H3 receptor modulating activity of the compound.

22. A method according to claim 21, wherein the step of incubating and the step of contacting are performed in the presence of a fixed concentration of H3 receptor agonist, wherein the fixed concentration is sufficient to stimulate GTP binding activity of the chimeric H3 receptor polypeptide in the control sample.

23. A method according to claim 21, wherein the step of incubating and the step of contacting are performed in the absence of added H3 receptor agonist.

24-25. (canceled)
